(19) United States
(12) Patent Application Publication
(10) Pub. No.: US 2012/0116059 A1
(43) Pub. Date: May 10, 2012

(54) Treatment For M Treatment For Multiple Sclerosis

(75) Inventors: Stefan Steidl, München (DE); Manuela Duerr, Nurtingen (DE); Elisabeth Thomassen-Wolf, Gauting (DE); Matthew Downham, Firenze (IT); Robert Friesen, Leiden (NL)

(73) Assignee: MORPHOSYS AG, Planegg-Martinsried (DE)

(21) Appl. No.: 13/318,149

(22) PCT Filed: May 4, 2010
(86) PCT No.: PCT/EP2010/056012

§ 371 Date: Jan. 17, 2012

Related U.S. Application Data
(60) Provisional application No. 61/175,471, filed on May 5, 2009.

(51) Int. Cl. C07K 16/24; A61P 25/00; C07K 16/28

(52) U.S. Cl. 530/389.2; 530/389.8

(57) ABSTRACT

The present invention relates to methods for the treatment and/or prophylaxis of multiple sclerosis (MS). Antagonists of GM-CSF, such as antibodies specific for GM-CSF or the GM-CSF receptor, are effective in the treatment and/or prophylaxis of multiple sclerosis.

### CROSS-REFERENCE TO RELATED APPLICATION

This application claims the benefit of U.S. provisional application Ser. No. 61/175,471 filed May 5, 2009, which is incorporated by reference in its entirety.

### FIELD OF THE INVENTION

The present invention relates generally to a method for the treatment and/or prophylaxis of multiple sclerosis (MS). In accordance with the present invention, an antagonist of GM-CSF can be effective in the treatment of multiple sclerosis. An antagonist of GM-CSF includes, but is not limited to, an antibody that is specific for GM-CSF or the GM-CSF receptor.

### BACKGROUND OF THE INVENTION

Multiple sclerosis (MS), also known as also known as disseminated sclerosis or encephalomyelitis disseminata, is an autoimmune disease in which the immune system attacks the central nervous system (CNS). Essentially, MS affects the ability of nerve cells in the brain and spinal cord to communicate with each other by damaging the myelin. When myelin is lost, the axons can no longer effectively conduct signals.

Four main subtypes of MS have been described (Neurology (1996) 46; 901-911; see also FIG. 1). The relapsing-remitting subtype is characterized by unpredictable relapses followed by periods of months to years of relative quiet (remission) with no new signs of disease activity. Secondary progressive MS describes those with initial relapsing-remitting MS, who then begin to have progressive neurologic decline between acute attacks without any definite periods of remission. The primary progressive subtype describes patients who never have remission after their initial MS symptoms. is characterized by progression of disability from onset, with no, or only occasional and minor, remissions and improvements. Finally, the progressive relapsing MS describes those individuals who, from onset, have a steady neurologic decline but also suffer clear superimposed attacks. Various borderline cases of MS exist as well.

Symptoms of MS are multi-facetted and usually appear in episodic acute periods of worsening (relapses), in a gradually-progressive deterioration of neurologic function, or in a combination of both. Common are sensorial, visual, cerebellar, and motor symptoms. MS patients can suffer from almost any neurological symptom or sign, including changes in sensation (hypoesthesia and paraesthesia), muscle weakness, muscle spasms, difficulty in moving; difficulties with coordination and balance; problems in speech or swallowing; visual problems; fatigue, acute or chronic pain, and bladder and bowel difficulties. Cognitive impairment of varying degrees and emotional symptoms of depression or unstable mood are also common.

MS usually appears in adults in their thirties, but it can also appear in children, and the primary progressive subtype is more common in people in their fifties. As with many autoimmune disorders, the disease is more common in women, and the trend may be increasing.

In MS, the immune system attacks the nervous system, possibly as a result of exposure to a molecule with a similar structure to one of its own. MS lesions most commonly involve white matter areas close to the ventricles of the cerebellum, brain stem, basal ganglia and spinal cord; and the optic nerve. The function of white matter cells is to carry signals between grey matter areas, where the processing is done, and the rest of the body. MS destroys oligodendrocytes, the cells responsible for creating and maintaining a fatty layer—known as the myelin sheath—which helps the neurons carry electrical signals. MS results in a thinning or complete loss of myelin and, as the disease advances, the cutting (transection) of the neuron's extensions or axons. A repair process, called remyelination, takes place in early phases of the disease, but the oligodendrocytes cannot completely rebuild the cell's myelin sheath. Repeated attacks lead to successively fewer effective remyelinations, until a scar-like plaque is built up around the damaged axons.

Apart from demyelination, the other pathologic hallmark of the disease is inflammation. T cells recognize myelin as foreign and attack it as if it were an invading virus. This triggers inflammatory processes, stimulating other immune cells and soluble factors like cytokines and antibodies.

Several therapies for multiple sclerosis exist, but there is no known cure. Different therapies are used for the management of acute attacks, to modify the disease and to manage the effects of MS. For the management of acute symptomatic attacks i.v. or oral administration of corticosteroids is the routine therapy. This treatment is effective in the short term for relieve of symptoms, but does not have a significant impact on long-term recovery of the patient. Numerous therapies for disease-modifying treatment are in use, depending on the exact nature and subtype of MS. Treatments for relapsing-remitting MS include interferons (interferon beta-1a and interferon beta-1b), glatiramer acetate (Copaxone; a mixture of polypeptides which may protect myelin proteins by substituting itself as the target of immune system attack), mitoxantrone (an immunosuppressant, also used in cancer chemotherapy) and natalizumab (Tysabri; a humanized monoclonal antibody the cellular adhesion molecule α4-integrin). Treatments for secondary progressive MS and progressive relapsing MS include mitoxantrone, natalizumab and interferon-beta-1b (betaferon). Other, mainly exploratory treatments are in use as well. MS is associated with a variety of symptoms and functional deficits that result in a range of progressive impairments and handicap. For the management of the effects of MS numerous therapies are known and used, as the specific case may require.

Some cytokines are known to be involved in multiple sclerosis, including granulocyte macrophage colony-stimulating factor (Ponomarev et al.; J Immunol (2007) 178; 39-48; McQualter et al.; J Exp Med. (2001) 194; 873-82). Granulocyte macrophage colony-stimulating factor (GM-CSF) is a cytokine that functions as a white blood cell growth factor. GM-CSF stimulates stem cells to produce granulocytes (neutrophils, eosinophils, and basophils) and monocytes. Monocytes exit the circulation and migrate into tissue, whereupon they mature into macrophages. It is, thus, part of the natural immune/inflammatory cascade, by which activation of a small number of macrophages can rapidly lead to an increase in their numbers, a process crucial for fighting infection. The active form of GM-CSF is found extracellularly as a soluble monomer. In particular, GM-CSF has been identified as an inflammatory mediator in autoimmune disorders, like rheumatoid arthritis (RA), leading to an increased production of pro-inflammatory cytokines, chemokines and proteases and, thereby, ultimately to articular destruction.

### SUMMARY OF THE INVENTION

The present invention demonstrates that antagonizing the effects of GM-CSF is a valid approach for the treatment of MS. In particular, antibodies against GM-CSF or its receptor are valid points of intervention in the treatment of MS. Accordingly, the invention provides, e.g., a method for the treatment of multiple sclerosis in a subject, said method comprising the step of administering an effective amount of a GM-CSF antagonist to said subject.

In another aspect, the present invention contemplates a method for the prophylaxis of multiple sclerosis in a subject, said method comprising the step of administering an effective amount of a GM-CSF antagonist to said subject.

In another aspect, the present invention is directed to a composition comprising a GM-CSF antagonist capable of antagonizing the pathophysiological role of GM-CSF in multiple sclerosis, said composition further comprising one or more pharmaceutically acceptable carriers and/or diluents.

In another aspect, the present invention is directed to a composition comprising a GM-CSF antagonist useful in the treatment of multiple sclerosis, said composition further comprising one or more pharmaceutically acceptable carriers and/or diluents.

In particular aspects of the present invention, the GM-CSF antagonist is an antibody specific for GM-CSF.

In alternative aspects of the present invention, the GM-CSF antagonist is an antibody specific for the GM-CSF receptor.

In other aspects, the present invention is directed to the use of a GM-CSF antagonist in the preparation of a medicament in the treatment of multiple sclerosis.

In other aspects, the present invention provides GM-CSF antagonists for the treatment of multiple sclerosis.

Throughout this specification, unless the context requires otherwise, the words “comprise”, “have” and “include” and their respective variations such as “comprises”, “comprising”, “has”, “having”, “includes” and “including” will be understood to imply the inclusion of a stated element or integer or group of elements or integers but not the exclusion of any other element or integer or group of elements or integers.

### BRIEF DESCRIPTION OF THE DRAWINGS

FIG. 1 depicts the progression types of the four main subtypes of MS.

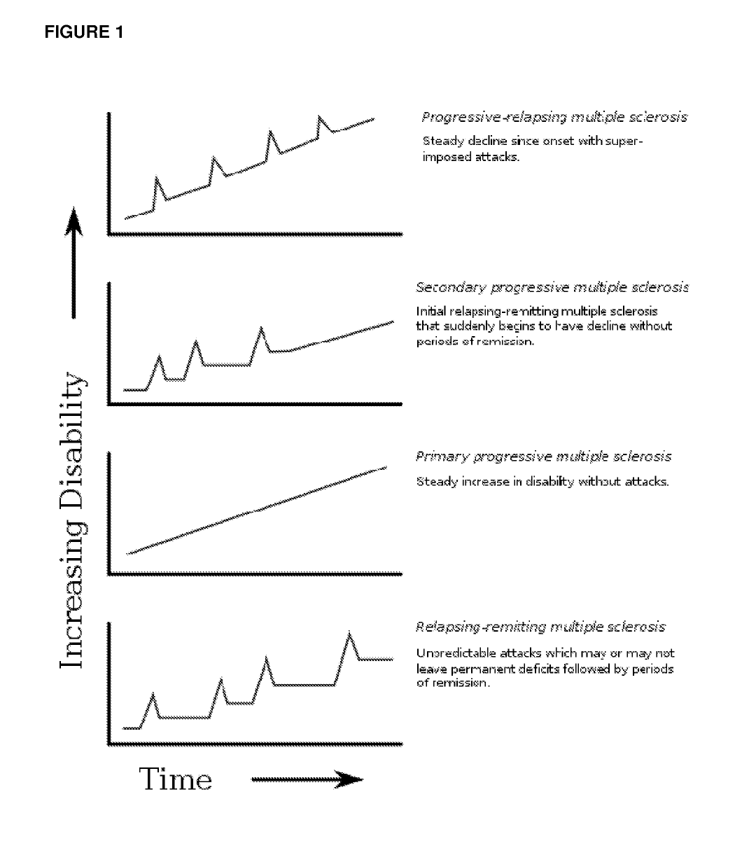

FIG. 2 shows the efficacy of a GM-CSF antagonist in a MOG-induced EAE model of MS (prophylactic treatment). Shown are the cumulative EAE scores for days 0-15. A: animals treated with the vehicle (PBS); B: animals treated with 10 mg/kg MOR-GM; C: animals treated with 20 mg/kg MOR-GM; D: animals treated with 50 mg/kg MOR-GM; E: animals treated with 0.5 mg/kg dexamethasone; F: animals treated with 50 mg/kg MOR-NOGM (isotype-control antibody); G: animals treated with 50 mg/kg MOR-GM with first treatment on day 14 (therapeutic treatment). *: P<0.05 as compare to the isotype-control antibody. $: P<0.05 as compare to PBS treatment.

FIG. 3 shows the delay of onset of disease is delayed upon treatment with MOR-GM. A: animals treated with the vehicle (PBS); B: animals treated with 10 mg/kg MOR-GM; C: animals treated with 20 mg/kg MOR-GM; D: animals treated with 50 mg/kg MOR-GM; E: animals treated with 0.5 mg/kg dexamethasone; F: animals treated with 50 mg/kg MOR-NOGM (isotype-control antibody); G: animals treated with 50 mg/kg MOR-GM with first treatment on day 14 (therapeutic treatment). *: P<0.05 as compare to the isotype-control antibody. $: P<0.10 as compare to PBS treatment.

FIG. 4 shows the infiltration by inflammatory cells in the sacral part of the spinal cord after treatment with compounds of the present invention. A: animals treated with 50 mg/kg MOR-NOGM (isotype-control antibody; prophylactic treatment); B: animals treated with 50 mg/kg MOR-GM (prophylactic treatment); C: animals treated with 50 mg/kg MOR-GM (therapeutic treatment); D: animals treated with 0.5 mg/kg dexamethasone (prophylactic treatment). Each data point indicates the scores of one individual animal. #: P<0.10 as compare to the isotype-control antibody. *: P<0.05 as compare to the isotype-control antibody.

FIG. 5 shows the extent of demyelination in the sacral part of the spinal cord. A: animals treated with 50 mg/kg MOR-NOGM (isotype-control antibody; prophylactic treatment); B: animals treated with 50 mg/kg MOR-GM (prophylactic treatment); C: animals treated with 50 mg/kg MOR-GM (therapeutic treatment); D: animals treated with 0.5 mg/kg dexamethasone (prophylactic treatment). Each data point indicates the scores of one individual animal. *: P<0.05 as compare to the isotype-control antibody.

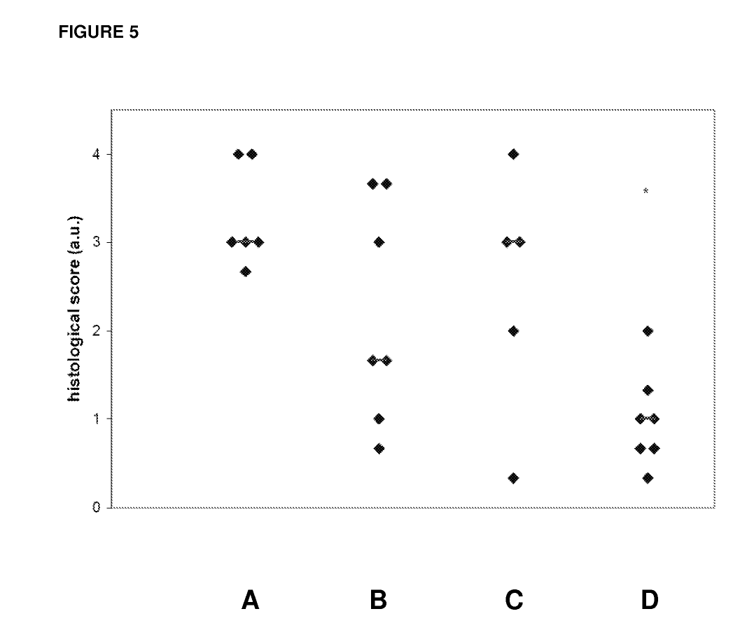

### DETAILED DESCRIPTION OF THE INVENTION

The present invention demonstrates that GM-CSF is a valid target for the treatment of MS. In this respect, the invention provides, in one aspect, methods of using a GM-CSF antagonist to bring about a prophylactic or therapeutic benefit in the field of MS.

The present invention provides therapeutic methods comprising the administration of a therapeutically effective amount of a GM-CSF antagonist to a subject in need of such treatment. A “therapeutically effective amount” or “effective amount”, as used herein, refers to the amount of a GM-CSF antagonist necessary to elicit the desired biological response. In accordance with the subject invention, the therapeutic effective amount is the amount of a GM-CSF antagonist necessary to treat and/or prevent multiple sclerosis.

“GM-CSF antagonists”, as used herein, includes GM-CSF antagonists in its broadest sense; any molecule which inhibits the activity or function of GM-CSF, or which by any other way exerts a therapeutic effect on GM-CSF is included. The term GM-CSF antagonists includes, but is not limited to, antibodies specifically binding to GM-CSF, inhibitory nucleic acids specific for GM-CSF or small organic molecules specific for GM-CSF. Also within the meaning of the term GM-CSF antagonist are antibodies specifically binding to the GM-CSF receptor, inhibitory nucleic acids specific for the GM-CSF receptor or small organic molecules specific for the GM-CSF receptor. The term GM-CSF antagonists also refers to non-antibody scaffold molecules, such as fibronectin scaffolds, ankyrins, maxybodies/avimers, protein A-derived molecules, anticalins, affilins, protein epitope mimetics (PEMs) or the like.

Inhibitory nucleic acids include, but are not limited to, antisense DNA, triplex-forming oligonucleotides, external guide sequences, siRNA and microRNA. Useful inhibitory nucleic acids include those that reduce the expression of RNA encoding GM-CSF by at least 20, 30, 40, 50, 60, 70, 80, 90 or 95 percent compared to controls. Inhibitory nucleic acids and methods of producing them are well known in the art. siRNA design software is available.

Small organic molecules (SMOLs) specific for GM-CSF or the GM-CSF receptor may be identified via natural product screening or screening of chemical libraries. Typically the molecular weight of SMOLs is below 500 Dalton, more typically from 160 to 480 Daltons. Other typical properties of SMOLs are one or more of the following:
The partition coefficient log P is in the range from −0.4 to +5.6
The molar refractivity is from 40 to 130
The number of atoms is from 20 to 70
   For reviews see Ghose et al. (1999) J Combin Chem: 1, 55-68 and Lipinski et al (1997) Adv Drug Del Rev: 23, 3-25.

Preferably, a GM-CSF antagonist for use in the present invention is an antibody specific for GM-CSF or specific for the GM-CSF receptor. Such an antibody may be of any type, such as a murine, a rat, a chimeric, a humanized or a human antibody. A “human” antibody or functional human antibody fragment is hereby defined as one that is not chimeric (e.g., not “humanized”) and not from (either in whole or in part) a non-human species. A human antibody or functional antibody fragment can be derived from a human or can be a synthetic human antibody. A “synthetic human antibody” is defined herein as an antibody having a sequence derived, in whole or in part, in silico from synthetic sequences that are based on the analysis of known human antibody sequences. In silico design of a human antibody sequence or fragment thereof can be achieved, for example, by analyzing a database of human antibody or antibody fragment sequences and devising a polypeptide sequence utilizing the data obtained therefrom. Another example of a human antibody or functional antibody fragment is one that is encoded by a nucleic acid isolated from a library of antibody sequences of human origin (i.e., such library being based on antibodies taken from a human natural source).

A “humanized antibody” or functional humanized antibody fragment is defined herein as one that is (i) derived from a non-human source (e.g., a transgenic mouse which bears a heterologous immune system), which antibody is based on a human germline sequence; or (ii) chimeric, wherein the variable domain is derived from a non-human origin and the constant domain is derived from a human origin or (iii) CDR-grafted, wherein the CDRs of the variable domain are from a non-human origin, while one or more frameworks of the variable domain are of human origin and the constant domain (if any) is of human origin.

The term “chimeric antibody” or functional chimeric antibody fragment is defined herein as an antibody molecule which has constant antibody regions derived from, or corresponding to, sequences found in one species and variable antibody regions derived from another species. Preferably, the constant antibody regions are derived from, or corresponding to, sequences found in humans, e.g. in the human germ line or somatic cells, and the variable antibody regions (e.g. VH, VL, CDR or FR regions) are derived from sequences found in a non-human animal, e.g. a mouse, rat, rabbit or hamster.

As used herein, an antibody “binds specifically to”, “specifically binds to”, is “specific to/for” or “specifically recognizes” an antigen (here, GM-CSF or, alternatively, the GM-CSF receptor) if such antibody is able to discriminate between such antigen and one or more reference antigen(s), since binding specificity is not an absolute, but a relative property. The reference antigen(s) may be one or more closely related antigen(s), which are used as reference points, e.g. IL3, IL5, IL-4, IL13 or M-CSF. In its most general form (and when no defined reference is mentioned), “specific binding” is referring to the ability of the antibody to discriminate between the antigen of interest and an unrelated antigen, as determined, for example, in accordance with one of the following methods. Such methods comprise, but are not limited to Western blots, ELISA-, RIA-, ECL-, IRMA-tests and peptide scans. For example, a standard ELISA assay can be carried out. The scoring may be carried out by standard color development (e.g. secondary antibody with horseradish peroxide and tetramethyl benzidine with hydrogenperoxide). The reaction in certain wells is scored by the optical density, for example, at 450 nm. Typical background (=negative reaction) may be 0.1 OD; typical positive reaction may be 1 OD. This means the difference positive/negative can be more than 10-fold. Typically, determination of binding specificity is performed by using not a single reference antigen, but a set of about three to five unrelated antigens, such as milk powder, BSA, transferrin or the like. Additionally, “specific binding” may relate to the ability of an antibody to discriminate between different parts of its target antigen, e.g. different domains or regions of GM-CSF or the GM-CSF receptor, or between one or more key amino acid residues or stretches of amino acid residues of GM-CSF or the GM-CSF receptor.

Also, as used herein, an “immunoglobulin” (Ig) hereby is defined as a protein belonging to the class IgG, IgM, IgE, IgA, or IgD (or any subclass thereof), and includes all conventionally known antibodies and functional fragments thereof. A “functional fragment” of an antibody/immunoglobulin hereby is defined as a fragment of an antibody/immunoglobulin (e.g., a variable region of an IgG) that retains the antigen-binding region. An “antigen-binding region” of an antibody typically is found in one or more hypervariable region(s) of an antibody, i.e., the CDR-1, -2, and/or -3 regions; however, the variable “framework” regions can also play an important role in antigen binding, such as by providing a scaffold for the CDRs. Preferably, the “antigen-binding region” comprises at least amino acid residues 4 to 103 of the variable light (VL) chain and 5 to 109 of the variable heavy (VH) chain, more preferably amino acid residues 3 to 107 of VL and 4 to 111 of VH, and particularly preferred are the complete VL and VH chains (amino acid positions 1 to 109 of VL and 1 to 113 of VH; numbering according to WO 97/08320). A preferred class of immunoglobulins for use in the present invention is IgG. “Functional fragments” of the invention include the domain of a F(ab′)₂ fragment, a Fab fragment, scFv or constructs comprising single immunoglobulin variable domains or single domain antibody polypeptides, e.g. single heavy chain variable domains or single light chain variable domains. The F(ab′)₂ or Fab may be engineered to minimize or completely remove the intermolecular disulphide interactions that occur between the C_{H1} and C_{L} domains.

An antibody of the invention may be derived from a recombinant antibody library that is based on amino acid sequences that have been designed in silico and encoded by nucleic acids that are synthetically created. In silico design of an antibody sequence is achieved, for example, by analyzing a database of human sequences and devising a polypeptide sequence utilizing the data obtained therefrom. Methods for designing and obtaining in silico-created sequences are described, for example, in Knappik et al., J. Mol. Biol. (2000) 296:57; Krebs et al., J. Immunol. Methods. (2001) 254:67, Rothe et al., J. Mol. Biol. (2008) 376:1182; and U.S. Pat. No. 6,300,064 issued to Knappik et al., which hereby are incorporated by reference in their entirety.

Any antibody specific for GM-CSF may be used with the present invention. Exemplary antibodies include antibodies comprising an amino acid sequence of a heavy chain variable region as depicted in SEQ ID No.:1 or an amino acid sequence of a light chain variable region as depicted in SEQ ID No.:2. Other exemplary antibodies include antibodies which are derived from antibodies comprising a heavy chain variable region as depicted in SEQ ID No.:1 or an amino acid sequence of a light chain variable region as depicted in SEQ ID No.:2. Yet other exemplary antibodies include antibodies which have the same specificity and/or bind to the same epitope as antibodies comprising a heavy chain variable region as depicted in SEQ ID No.:1 or an amino acid sequence of a light chain variable region as depicted in SEQ ID No.:2. Yet other exemplary antibodies include antibodies which comprise a heavy chain variable region which is at least 70%, at least 80%, at least 90% or at least 95% homologous to the sequence depicted in SEQ ID No.:1. Yet other exemplary antibodies include antibodies which comprise a light chain variable region which is at least 70%, at least 80%, at least 90% or at least 95% homologous to the sequence depicted in SEQ ID No.:2.

SEQ ID No. 1:
Met Glu Leu Ile Met Leu Phe Leu Leu Ser Gly Thr Ala Gly Val His
Ser Glu Val Gln Leu Gln Gin Ser Gly Pro Glu Leu Val Lys Pro Gly
Ala Ser Val Lys Ile Ser Cys Lys Ala Ser Gly Tyr Thr Phe Thr Asp
Tyr Asn Ile His Trp Val Lys Gln Ser His Gly Lys Set Leu Asp Trp
Ile Gly Tyr Ile Ala Pro Tyr Ser Gly Gly Thr Gly Tyr Asn Gln Glu
Phe Lys Asn Arg Ala Thr Leu Thr Val Asp Lys Ser Ser Ser Thr Ala
Tyr Met Glu Leu Arg Ser Leu Thr Ser Asp Asp Ser Ala Val Tyr Tyr
Cys Ala Arg Arg Asp Arg Phe Pro Tyr Tyr Phe Asp Tyr Trp Gly Gln Gly
Thr Thr Leu Arg Val Ser Ser Val Ser Gly Ser
SEQ ID No. 2:
Met Gly Phe Lys Met Glu Ser Gln Ile Gln Val Phe Val Tyr Met Leu
Leu Trp Leu Ser Gly Val Asp Gly Asp Ile Val Met Ile Gln Ser Gln
Lys Phe Val Ser Thr Ser Val Gly Asp Arg Val Asn Ile Thr Cys Lys
Ala Ser Gln Asn Val Gly Ser Asn Val Ala Trp Leu Gln Gln Lys Pro
Gly Gln Ser Pro Lys Thr Leu Ile Tyr Ser Ala Ser Tyr Arg Ser Gly
Arg Val Pro Asp Arg Phe Thr Gly Set Gly Ser Gly Thr Asp Phe Ile
Leu Thr Ile Thr Thr Val Gln Ser Glu Asp Leu Ala Glu Tyr Phe Cys
Gln Gln Phe Asn Arg Ser Pro Leu Thr Phe Gly Ser Gly Thr Lys Leu
Glu Leu Lys Arg Ala Asp Ala Ala Pro Thr Val Ser Ile Phe Pro Pro
Ser Ser Lys Gly Glu Phe

Alternative exemplary antibodies that can be used in the present invention are antibodies comprising an amino acid sequence of a heavy chain variable region as depicted in SEQ ID No.:3 or an amino acid sequence of a light chain variable region as depicted in SEQ ID No.:4. Other exemplary antibodies include antibodies which are derived from antibodies comprising a heavy chain variable region as depicted in SEQ ID No.:3 or an amino acid sequence of a light chain variable region as depicted in SEQ ID No.:4. Yet other exemplary antibodies include antibodies which have the same specificity and/or bind to the same epitope as antibodies comprising a heavy chain variable region as depicted in SEQ ID No.:3 or an amino acid sequence of a light chain variable region as depicted in SEQ ID No.:4. Yet other exemplary antibodies include antibodies which comprise a heavy chain variable region which is at least 70%, at least 80%, at least 90% or at least 95% homologous to the sequence depicted in SEQ ID No.:3. Yet other exemplary antibodies include antibodies which comprise a light chain variable region which is at least 70%, at least 80%, at least 90% or at least 95% homologous to the sequence depicted in SEQ ID No.:4.

SEQ ID NO. 3:
QVQLVESGGGLVQPGGSLRLSCAASGFTFSSYWMNWVRQAPGKGLEWVSGIENKYAGGA
TYYAASVKGRFTISRDNSKNTLYLQMNSLRAEDTAVYYCARGFGTDFWGQGTLVTVSS
SEQ ID NO. 4:
DIELTQPPSVSVAPGQTARISCSGDSIGKKYAYWYQQKPGQAPVLVIYKKRPSGIPERFSGSNS
GNTATLTISGTQAEDEADYYCSAWGDKGMVFGGGTKLTVLGQ

Alternative exemplary antibodies that can be used in the present invention are antibodies comprising a H-CDR3 sequence selected from:

(SEQ ID NO. 5)
Ser Gly Leu Ile Phe Asp Tyr Trp Leu Asp,
1 5 10
(SEQ ID NO. 6)
Ser Gly Leu Ile Ile Asp Ala Leu Ser Pro,
1 5 10
(SEQ ID NO. 7)
Thr Ser Leu Met Ser Ile Tyr Phe Asp Tyr,
1 5 10
(SEQ ID NO. 8)
Ser Gly Leu Leu Phe Leu Tyr Phe Asp Tyr,
1 5 10
(SEQ ID NO. 9)
Ser Gly Leu Ile Asn Leu Gly Met His Pro,
1 5 10
(SEQ ID NO. 10)
Ser Gly Leu Ile Phe Asp Ala Leu Arg Asp,
1 5 10
(SEQ ID NO. 11)
Ser Gly Leu Ile Phe Asp Lys Leu Thr Ser,
1 5 10
(SEQ ID NO. 12)
Ser Gly Leu Ile Asn Leu His Phe Asp Thr,
1 5 10
(SEQ ID NO. 13)
Ser Thr His Phe Ser Ala Tyr Phe Asp Tyr,
1 5 10
(SEQ ID NO. 14)
Ser Gly Leu Ile Met Asp Lys Leu Asp Asn,
1 5 10
(SEQ ID NO. 15)
Ser Gly Leu Ile Ile Asp Asn Leu Asn Pro,
1 5 10
and
(SEQ ID NO. 16)
Ser Gly Leu Ile Ala Val Tyr Phe Asp Tyr.
1 5 10

Preferably, the antibodies comprising a H-CDR3 sequence selected from any one of SEQ ID NOs. 5-16, additionally comprise the following H-CDR1 sequence:

(SEQ ID NO. 16)
Asp Tyr Leu Leu His,
1 5

and/or the following H-CDR2 sequence:

(SEQ ID NO. 17)
Trp Leu Asn Pro Tyr Ser Gly Asp Thr Asn Tyr Ala Gln Lys Phe Gln
1 5 10 15
Gly,

and/or the following L-CDR1 sequence:

(SEQ ID NO. 18)
Arg Ala Ser Gln Asn Ile Arg Asn Ile Leu Asn,
1 5 10

and/or the following L-CDR2 sequence:

(SEQ ID NO. 19)
Ala Ala Ser Asn Leu Gln Ser,
1 5

and/or the following L-CDR3 sequence:

(SEQ ID NO. 20)
Gln Gln Ser Tyr Ser Met Pro Arg Thr.
1 5

Alternative exemplary antibodies that can be used in the present invention are antibodies comprising the following L-CDR1 sequence:

(SEQ ID NO. 21)
Arg Ala Ser His Arg Val Ser Ser Asn Tyr Leu Ala,
1 5 10

and/or the following L-CDR2 sequence:

(SEQ ID NO. 22)
Gly Ala Ser Asn Arg Ala Thr,
1 5

and/or the following L-CDR3 sequence:

(SEQ ID NO. 23)
Gln Gln Tyr Ala Ser Ser Pro Val Thr,
1 5

and/or the following H-CDR1 sequence:

(SEQ ID NO. 24)
Gly Tyr Ile Phe Pro Thr Phe Ala Leu His,
1 5 10

and/or the following H-CDR2 sequence:

(SEQ ID NO. 25)
Ser Ile Asn Thr Ala Ser Gly Lys Thr Lys Phe Ser Thr Lys Phe Gln,
1 5 10 15

and/or the following H-CDR3 sequence:

(SEQ ID NO. 26)
Asp Arg Phe Gln Asn Ile Met Ala Thr Ile Leu Asp Val.
1 5 10

Preferably said antibody comprise all the CRDs of SEQ ID NOs. 21-26.

The GM-CSF receptor is a member of the haematopoietin receptor superfamily. It is heterodimeric, consisting of an alpha and a beta subunit. The alpha subunit is highly specific for GM-CSF whereas the beta subunit is shared with other cytokine receptors, including IL3 and IL5. This is reflected in a broader tissue distribution of the beta receptor subunit. The alpha subunit, GM-CSFR α, is primarily expressed on myeloid cells and non-haematopoetic cells, such as neutrophils, macrophages, eosinophils, dendritic cells, endothelial cells and respiratory epithelial cells. Full length GM-CSFR α is a 400 amino acid type I membrane glycoprotein that belongs to the type I cytokine receptor family, and consists of a 22 amino acid signal peptide (positions 1-22), a 298 amino acid extracellular domain (positions 23-320), a transmembrane domain from positions 321-345 and a short 55 amino acid intra-cellular domain. The signal peptide is cleaved to provide the mature form of GM-CSFR α as a 378 amino acid protein. cDNA clones of the human and murine GM-CSFR α are available and, at the protein level, the receptor subunits have 36% identity. GM-CSF is able to bind with relatively low affinity to the α subunit alone (Kd 1-5 nM) but not at all to the β subunit alone. However, the presence of both α and β subunits results in a high affinity ligand-receptor complex (Kd>>100 μM). GM-CSF signalling occurs through its initial binding to the GM-CSFR α chain and then cross-linking with a larger subunit the common β chain to generate the high affinity interaction, which phosphorylates the JAK-STAT pathway.

Any antibody specific for GM-CSF receptor may be used with the present invention. Exemplary antibodies include antibodies comprising an amino acid sequence of a H-CDR3 sequence depicted in any one of SEQ ID No's.:27-45. Other exemplary antibodies include antibodies which are derived from antibodies comprising an amino acid sequence of a H-CDR3 sequence depicted in any one of SEQ ID No's.:27-45. Yet other exemplary antibodies include antibodies which have the same specificity and/or bind to the same epitope as antibodies comprising an amino acid sequence of a H-CDR3 sequence depicted in any one of SEQ ID No's.:27-45. Yet other exemplary antibodies include antibodies which comprise a H-CDR3 sequence which is at least 70%, at least 80%, at least 90% or at least 95% homologous to the H-CDR3 sequence depicted in any one of SEQ ID No's.:27-45.

```
SEQ ID No: 27:
Val Gly Ser Phe Ser Gly Ile Ala Tyr Arg Pro
                                5 10
SEQ ID No: 28:
Val Gly Ser Phe Ser Gly Pro Ala Leu Arg Pro
                                5 10
SEQ ID No: 29:
Val Gly Ser Phe Ser Pro Pro Thr Tyr Gly Tyr
                                5 10
SEQ ID No: 30:
<400> 45
Val Gly Ser Phe Ser Gly Tyr Pro Tyr Arg Pro
                5 10
SEQ ID No: 31:
Val Gly Ser Phe Ser Pro Leu Thr Leu Gly Leu
                                5 10
SEQ ID No: 32:
Val Gly Ser Phe Ser Gly Pro Val Tyr Gly Leu
                                5 10
SEQ ID No: 33:
Val Gly Ser Phe Ser Pro Pro Ala Tyr Arg Pro
                                5 10
SEQ ID No: 34:
Val Gly Ser Phe Ser Pro Val Thr Tyr Gly Leu
                                5 10
SEQ ID No: 35:
Val Gly Ser Phe Ser Gly Leu Ala Tyr Arg Pro
                                5 10
SEQ ID No: 36:
Val Gly Ser Phe Ser Pro Ile Thr Tyr Gly Leu
                                5 10
SEQ ID No: 37:
Val Gly Ser Phe Ser Gly Trp Ala Phe Asp Tyr
                                5 10
SEQ ID No: 38:
Val Gly Ser Phe Ser Gly Trp Ala Phe Asp Tyr
                                5 10
SEQ ID No: 39:
Leu Gly Ser Val Thr Ala Trp Ala Phe Asp Tyr
                                5 10
SEQ ID No: 40:
Ala Gly Ser Ile Pro Gly Trp Ala Phe Asp Tyr
                                5 10
SEQ ID No: 41:
Val Gly Ser Phe Ser Pro Leu Thr Met Gly Leu
                                5 10
SEQ ID No: 42:
Val Gly Ser Phe Ser Pro Leu Thr Met Gly Leu
                                5 10
SEQ ID No: 43:
Val Gly Ser Phe Ser Gly Pro Ala Leu His Leu
                                5 10
SEQ ID No: 44:
Val Gly Ser Val Ser Arg Ile Thr Tyr Gly Phe
                                5 10
SEQ ID No: 45:
Val Gly Ser Phe Ser Pro Leu Thr Leu Gly Leu
                                5 10
```

Another antibodies specific for GM-CSF that may be used with the present invention include antibodies comprising an amino acid sequence of a H-CDR3 sequence depicted in any one of SEQ ID No's.:46-56. Other exemplary antibodies include antibodies which are derived from antibodies comprising an amino acid sequence of a H-CDR3 sequence depicted in any one of SEQ ID No's.: 46-56. Yet other exemplary antibodies include antibodies which have the same specificity and/or bind to the same epitope as antibodies comprising an amino acid sequence of a H-CDR3 sequence depicted in any one of SEQ ID No's.: 46-56. Yet other exemplary antibodies include antibodies which comprise a H-CDR3 sequence which is at least 70%, at least 80%, at least 90% or at least 95% homologous to the H-CDR3 sequence depicted in any one of SEQ ID No's.: 46-56.

SEQ ID No: 46:
EGGYSYGYFDY
SEQ ID No: 47:
DKWLDGFDY
SEQ ID No: 48:
DRWLDAFDI
SEQ ID No: 49:
APYDWTFDY
SEQ ID No: 50:
DRWLDAFEI
SEQ ID No: 51:
QRYYYSMDV
SEQ ID No: 52:
RPWELPFDY
SEQ ID No: 53:
NGDYVFTYFDY
SEQ ID No: 54:
FGYFGYYFDY
SEQ ID No: 55:
DPYTSGFDY
SEQ ID No: 56:
EDTAMDYFDY

Compositions of the invention may be used for therapeutic or prophylactic applications. The invention, therefore, includes a pharmaceutical composition containing an inventive antibody (or functional antibody fragment) and a pharmaceutically acceptable carrier or excipient therefor. In a related aspect, the invention provides a method for treating multiple sclerosis. Such method contains the steps of administering to a subject in need thereof an effective amount of the pharmaceutical composition that contains an inventive antibody as described or contemplated herein.

In certain aspects, the present invention provides methods for the treatment of multiple sclerosis in a subject, said method comprising the step of administering a GM-CSF antagonist to said subject. “Subject”, as used in this context refers to any mammal, including rodents, such as mouse or rat, and primates, such as cynomolgus monkey (Macaca fascicularis), rhesus monkey (Macaca mulatta) or humans (Homo sapiens). Preferably the subject is a primate, most preferably a human.

In certain aspects, the present invention provides methods for the treatment of multiple sclerosis, said method comprising the step of administering to a subject a GM-CSF antagonist, wherein said GM-CSF antagonist can bind to GM-CSF with an affinity of about less than 100 nM, more preferably less than about 60 nM, and still more preferably less than about 30 nM. Further preferred are antibodies that bind to GM-CSF with an affinity of less than about 10 nM, and more preferably less than about 3 nM.

In certain aspects, the present invention provides methods for the treatment of multiple sclerosis, said method comprising the step of administering to a subject a GM-CSF antagonist, wherein said GM-CSF antagonist competes for binding to GM-CSF with an antibody, wherein the heavy chain of said antibody comprises the amino acid sequence of SEQ ID No.:3. In alternative aspects, the present invention provides methods for the treatment of multiple sclerosis, said method comprising the step of administering to a subject a GM-CSF antagonist, wherein said GM-CSF antagonist competes for binding to GM-CSF with an antibody, wherein the light chain of said antibody specific for GM-CSF comprises the amino acid sequence of SEQ ID No.:4.

In certain aspects, the present invention provides methods for the treatment of multiple sclerosis, said method comprising the step of administering to a subject a GM-CSF antagonist, wherein said GM-CSF antagonist is an antibody specific for GM-CSF and wherein said antibody specific for GM-CSF is cross-reactive with rat and/or rhesus (macaca) GM-CSF, as determined by solution equilibrium titration (SET), and/or TF1 proliferation assay.

In certain aspect, the present invention provides a composition comprising a GM-CSF antagonist capable of antagonizing the pathophysiological role of GM-CSF in multiple sclerosis, said composition further comprising one or more pharmaceutically acceptable carriers and/or diluents. Anti-GM-CSF antibodies of the present invention may antagonize any of the roles of GM-CSF in multiple sclerosis.

In certain aspects, the present invention provides a composition comprising a GM-CSF antagonist capable of reducing demyelination of the myelin sheet, said composition further comprising one or more pharmaceutically acceptable carriers and/or diluents.

In certain aspects, the present invention provides a composition comprising a GM-CSF antagonist capable of reducing the influx of inflammatory cells into the spinal cord, said composition further comprising one or more pharmaceutically acceptable carriers and/or diluents.

In certain aspects, the present invention provides a method for the treatment or prophylaxis of multiple sclerosis in a subject, comprising the step of administering to the subject an effective amount of an antagonist of GM-CSF, wherein said administration delays the onset of multiple sclerosis.

In another aspect, the present invention provides a method for the prophylaxis of multiple sclerosis in a subject, said method comprising administering a GM-CSF antagonist to said subject. “Prophylaxis” as used in this context refers to methods which aim to prevent the onset of a disease or which delay the onset of a disease.

In certain aspects, the present invention provides a method for the treatment or prophylaxis of multiple sclerosis in a subject, comprising the step of administering to the subject an effective amount of an antagonist of GM-CSF, wherein said administration reduces proliferation of T cells.

In certain aspects, the present invention provides a method for the treatment or prophylaxis of multiple sclerosis in a subject, comprising the step of administering to the subject an effective amount of an antagonist of GM-CSF, wherein said administration reduces the release of IL17 by T cells.

Assays to measure and quantify T cell proliferation and the release of IL17 are known in the art.

In certain aspects, the present invention provides a composition comprising a GM-CSF antagonist useful in the treatment of multiple sclerosis, said composition further comprising one or more pharmaceutically acceptable carriers and/or diluents.

In other aspects, the present invention provides the use of a GM-CSF antagonist in the preparation of a medicament in the treatment of multiple sclerosis.

In other aspects, the present invention provides GM-CSF antagonists for the treatment of multiple sclerosis.

In particular aspects, the GM-CSF antagonists of the present invention are administered subcutaneously. In other aspects, the GM-CSF antagonists of the present invention are administered intraspinally. GM-CSF antagonists may be particular effect for the treatment of multiple sclerosis when administered subcutaneously or intraspinally.

The compositions of the present invention are preferably pharmaceutical compositions comprising a GM-CSF antagonist and a pharmaceutically acceptable carrier, diluent or excipient, for the treatment of multiple sclerosis. Such carriers, diluents and excipients are well known in the art, and the skilled artisan will find a formulation and a route of administration best suited to treat a subject with the GM-CSF antagonists of the present invention.

In certain aspects, the present invention provides a method for the treatment or prophylaxis of multiple sclerosis in a subject, comprising the step of administering to the subject an effective amount of an antagonist of GM-CSF. In certain aspects said subject is a human. In alternative aspects said subject is a rodent, such as a rat or a mouse.

In certain aspects, said antagonist of GM-CSF is an antibody specific for GM-CSF. In particular aspects, the variable heavy chain of said antibody specific for GM-CSF comprises the amino acid sequence of SEQ ID No.:3. In other particular aspects, the variable light chain of said antibody specific for GM-CSF comprises the amino acid sequence of SEQ ID No.:4.

In certain aspects, said antagonist of GM-CSF is an antibody specific for the GM-CSF receptor.

In certain aspects, said administration of an antagonist of GM-CSF reduces demyelination of the myelin sheet.

In other aspects, said administration of an antagonist of GM-CSF reduces the influx of inflammatory cells into the spinal cord.

In yet other aspects, said administration of an antagonist of GM-CSF reduces the proliferation of T cells.

In yet other aspects, said administration of an antagonist of GM-CSF reduces the release of IL17 by T cells.

In yet other aspects, said administration delays the onset of multiple sclerosis.

In certain aspects, said antagonist of GM-CSF is administered subcutaneously or intraspinally.

In certain aspects, the present invention provides an antagonist of GM-CSF for use in the treatment or prophylaxis of multiple sclerosis. In certain aspects, said treatment or prophylaxis comprises the step of administering to a subject an effective amount of the antagonist of GM-CSF. In certain aspects, said subject is a human. In alternative aspects, said subject is a rodent, such as a rat or a mouse.

In certain aspects, said antagonist of GM-CSF is an antibody specific for GM-CSF. In certain aspects, the variable heavy chain of said antibody specific for GM-CSF comprises the amino acid sequence of SEQ ID No.:3. In certain aspects, the variable light chain of said antibody specific for GM-CSF comprises the amino acid sequence of SEQ ID No.:4.

In certain aspects, said antagonist of GM-CSF is an antibody specific for the GM-CSF receptor.

In certain aspects, the treatment or prophylaxis with said antagonist of GM-CSF reduces the demyelination of the myelin sheet.

In other aspects, the treatment or prophylaxis with said antagonist of GM-CSF reduces the influx of inflammatory cells into the spinal cord.

In other aspects, the treatment or prophylaxis with said antagonist of GM-CSF reduces the proliferation of T cells.

In other aspects, the treatment or prophylaxis with said antagonist of GM-CSF reduces the release of IL17 by T cells.

In other aspects, the treatment or prophylaxis with said antagonist of GM-CSF delays the onset of multiple sclerosis.

In certain aspects, said antagonist of GM-CSF is administered subcutaneously or intraspinally.

### EXAMPLES

### Example 1

### Exemplary antibodies and animals used in the present invention

MOR-GM was used as an exemplary GM-CSF antagonist in the present invention. MOR-GM is a fully human GM-CSF-specific antibody (WO 06/122797). The heavy chain variable region of MOR-GM is shown in SEQ ID No.:3, the light chain variable region in SEQ ID No.:4.

Antibody 22E9, an anti mouse GM-CSF antibody, was used in other experiments (AbD Serotec, Martinsried/Germany; Cat. No. 1023501).

Evidently, any other GM-CSF antagonist, for example any antibody comprising an amino acid stretch selected from SEQ ID No.s:1-45 could be used in accordance with the present invention.

Male Dark Agouti rats, 7-8 weeks old (Harlan Laboratories, Inc., Indianapolis/IN) were housed under clean conventional conditions at 21±3° C., relative humidity of 40-70% and a light/dark cycle of 12 hours. Rats were housed in pairs and had free access to rodent chow diet (SSNIFF, Bio-Services, The Netherlands). Individual animals were identified by marking the tail. Before start of the experiment, rats were handled for a 4-week period. Rats were assigned to groups by randomization before initiation of the experiment. At the start of the experiments rats were at an age of 11-12 weeks and had a weight of 200-250 grams.

### Example 2

### Therapeutic effectiveness of GM-CSF antagonists in a MOG-induced EAE model of MS

To induce experimental autoimmune encephalomyelitis (EAE) male DA rats were immunized in two sites flanking the dorsal base of the tail by intradermal injection with 15 μg of recombinant myelin-oligodendrocyte-glycoprotein (rMOG) emulsified in 200 μl of a 1:1 mixture of Freund's incomplete adjuvant (IFA) and 10 mM NaAc, pH 3.0. To facilitate immunization, rats were anesthetized by inhalation of 2-4% isoflurane in a mixture of oxygen an N₂O.

The effects of intraperitoneal administrations of the test compound MOR-GM were tested in comparison with vehicle (PBS) treatment and in comparison with a group treated with the non-specific/irrelevant isotype control antibody MOR-NOGM (50 mg/kg). Prophylactic treatment with compound MOR-GM was tested at three dosages, namely 10, 20 and 50 mg/kg. The compound was administered on days 7, 10, 14, 17 and 21. Furthermore, the efficacy of the compound (50 mg/kg) was tested at a regimen in which the first treatment was started after disease onset. In this case, the compound was administered on days 14, 17 and 21. Positive control groups comprised rats treated daily from day 9 onwards by i.p. administration of dexamethasone (0.5 mg/kg)

Each experimental group comprised 12 animals. Blood samples were collected from the tail veins on days 17 and 21 (before treatment) and at endpoint. The body weight of each individual rat was measured daily.

EAE of the rats was evaluated daily using the following disability scoring system:
0: no disease
0.5: tail paresis or partial paralysis
1: complete tail paralysis
2 hind limb weakness or partial paralysis
2.5: as 2, but with additional involvement of the front paws
3: complete paralysis of hind limbs and/or lower part of the body
3.5: as 3, but with additional involvement of the front paws
4: death due to EAE

Rats that needed to be euthanized due to EAE associated co-morbidity were assigned a value of 3.5 on the day of euthanasia, and a score of 4 on all subsequent days during the entire monitoring period.

“Maximal clinical score” refers to the highest EAE score of each rat during an experimental period

The “cumulative score” is the sum of all EAE scores for a given rat during a defined time period (area under the curve). The cumulative scores were calculated for the entire follow-up period, for the first disease phase (day 0-15) and for the relapse phase (day 16-end).

The “day of onset” refers to the first of three consecutive days on which a cumulative score of at least 3 was reached.

Histological analysis: Formalin-fixated tissues were embedded in paraffin and 5 μm tissue sections were stained with hematoxylin/eosin to enable semi-quantitative grading of infiltration of inflammatory cells, or stained with Luxol fast blue according to Klüver-Barrera for myelin staining. The extent of infiltration by inflammatory cells and demyelination were assessed in a semi-quantitative fashion on three non-serial (separated by 100 μm) sections from the sacral part of the spinal cord. Histological grading was done employing the scoring systems as described in the tables below. Histological grading was performed in a blinded fashion.

Histological scoring system for semi-quantitative grading of infiltration of inflammatory cells into spinal cord tissue:

| Score | Criteria |
|---|---|
| 1 | Few areas (1-5) with mild perivascular cuffing of inflammatory |
| | cells |
| 2 | Moderate number of areas (5-10) with perivascular cuffing of |
| | inflammatory cells |
| 3 | Moderate number of areas (5-10) with perivascular cuffing and |
| | infiltration of parenchyma by inflammatory cells |
| 4 | Numerous areas (>10) with perivasular cuffing and extensive |
| | infiltration of parenchyma by inflammatory cells |

Histological scoring system for semi-quantitative grading of demyelination of spinal cord tissue:

| Score | Criteria |
|---|---|
| 1 | Few areas (1-5) with mild demyelination |
| 2 | Moderate number of areas (5-10) with mild demyelination |
| 3 | Moderate number of areas (5-10) with extensive demyelination |
| 4 | Numerous areas (>10) with extensive demyelination |

All statistical analyses were performed using the statistical software program SPSS 14 for Windows (SPSS Inc., Chicago, Ill., USA). For multiple group comparison of day of onset, maximal EAE score, cumulative score and the maximal weight loss, the Kruskal-Wallis test was applied, followed by a post-hoc Mann-Whitney U-test to determine which groups differed significantly from the vehicle-treated groups. Disease progression was tested with the General Linear Method, followed by ANOVA and a post-hoc LSD-test to determine on which days the treatments were significantly different. Kaplan-Meier analysis using the Mantel Cox test was used for multiple group comparison of the survival rate and subsequent post-hoc analysis.

Control Treatment with Vehicle (PBS; Negative Control)

Animals were treated on days 7, 10, 14, 17 and 21 by intraperitoneal administration of PBS. All animals developed EAE. The first signs of disease were observed on day 9. The mean day of onset was 10.1±0.7. The first bout peaked around day 11-13, the second bout around day 21. The mean maximal EAE score of this group was 3.5±0.6 and the mean cumulative EAE score from day 0-24 was 38.7±9.8. During the first phase of the disease (days 0 to day 15) the mean cumulative score was 11.7±2.7, during the relapse phase 26.9±7.6. All animals showed weight loss in association with signs of paralysis. The mean maximal percentage of weight loss was 21.1±5.7%.

Control Treatment with an Isotype-Control Antibody (MOR-NOGM; Negative Control)

Animals were treated on days 7, 10, 14, 17 and 21 with the isotype control antibody MOR-NOGM, at a dosage of 50 mg/kg. All animals in this group developed EAE. None of the observed parameters were statistically significantly different from the observations in the vehicle (PBS) treated group. The mean day of onset was 9.8±0.8. The mean maximal EAE score was 3.4±0.7. The cumulative EAE scores were comparable with the scores of the vehicle treated groups. The cumulative EAE score for the entire experimental period was 36.7±10.9. The cumulative for the first disease phase was 13.3±3.1 and for the relapse phase 23.5±8.0. The maximal weight loss was 19.6±5.6%. Animals showed substantial infiltration of the sacral part of the spinal cord by inflammatory cells along with extensive demyelination.

Control Treatment with Dexamethasone (Positive Control)

Animals were intraperitoneally treated with 0.5 mg/kg dexamethasone daily from day 9 onwards. Only 9 out of 12 animals developed EAE. Treatment did have an effect on the disease severity. The maximal EAE score was significantly reduced to 1.7±1.0 (p=0.001). The mean cumulative EAE score was reduced to 13.1±13.6 (p<0.005). The cumulative score during the first disease phase was reduced to 6.0±5.7 (p=0.010) and to 7.1±9.3 for the relapse phase (p<0.0005). Despite inhibition of EAE, dexamethasone did not affect loss of body weight. The extent of infiltration of inflammatory cells in the sacral part of the spinal cord was diminished as compared to rats treated with the isotype-control antibody MOR-NOGM (p=0.003). Likewise, the degree of demyelination was significantly lower (p=0.002).

Treatment with MOR-GM from Day 7 Onwards (Prophylactic Treatment)

Animals were treated on days 7, 10, 14, 17 and 21 intraperitoneally with MOR-GM at a dose of 10, 20 or 50 mg/kg. All animals did develop EAE. As compared to the treatment with the isotype-control antibody (MOR-NOGM), the day of disease onset was significantly delayed by 2 days by treatment with 50 mg/kg MOR-GM (disease onset: 11.8±3.0 days; p=0.047). There is also a clear trend towards reduction of the cumulative EAE score. The cumulative EAE score from day 0-24 was 27.7±12.3 (p=0.094). The cumulative EAE score during the initial phase of disease (day 0-15) was 8.7±4.9. This is significantly reduced as compared to the isotype treated control group (p=0.040). Treatment had no effect on the maximal weight loss. Treatment with 50 mg/kg MOR-GM reduced the daily mean cumulative score (p=0.022). When evaluated on a day-to-day basis, the mean cumulative scores were significantly reduced from day 11 until day.

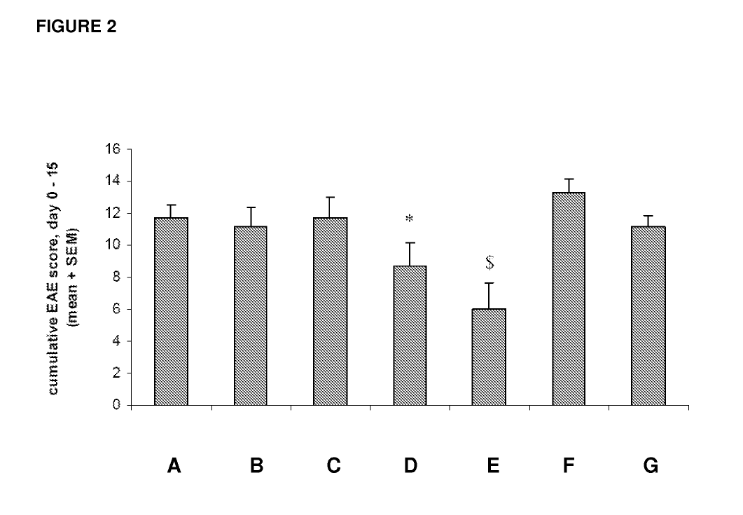

Results are depicted in FIG. 2. Prophylactic treatment with MOR-GM (50 mg/kg) showed a strong and significant reduction of the cumulative EAE score on days 0-15. Results were particular significant for the first bout (days 0-15).

Treatment with MOR-GM from Day 14 Onwards (Therapeutic Treatment)

Animals received the first treatment with 50 mg/kg MOR-GM (intraperitoneally) on day 14, i.e. four days after the onset of EAE. Further treatment was performed on days 17 and 21. This treatment regimen did not have a statistically significant effect on the maximal EAE score or the cumulative EAE scores. However, the cumulative score over time showed a less pronounced increase in the clinical score compared to the isotype control antibody upon start of therapeutic treatment on day 14. Maximal loss of body weight was not influenced.

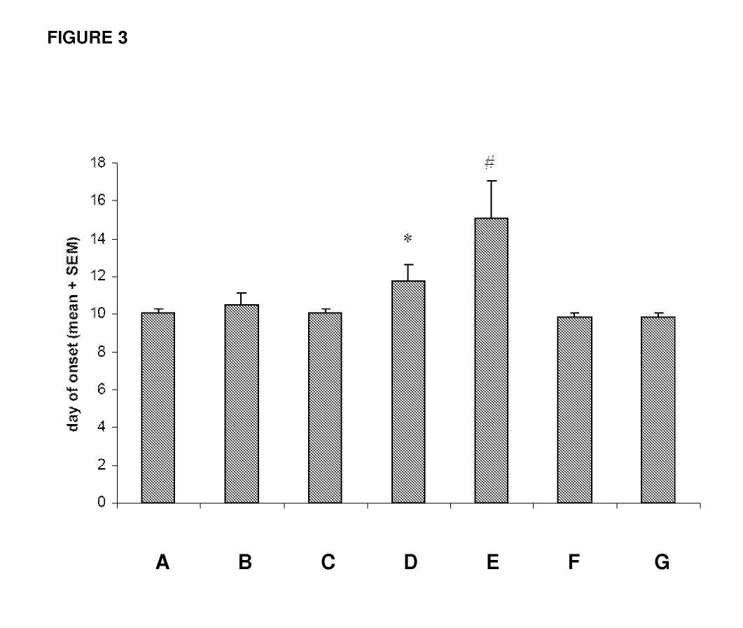

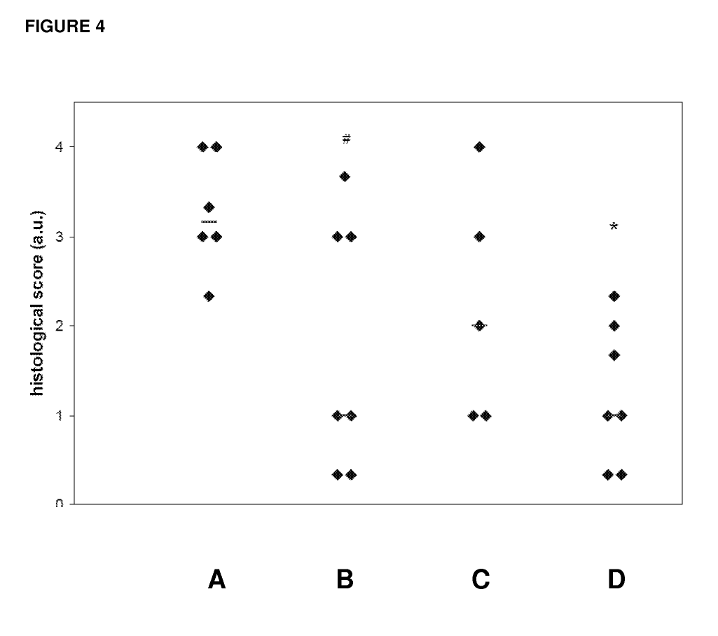

FIG. 3 shows that the onset of disease is delayed upon administration of MOR-GM at a concentration of 50 mg/kg in a prophylactic treatment (P<0.10). Histological findings are depicted in FIGS. 4 (inflammation) and 5 (demyelination). Results clearly show that GM-CSF antagonists are able to reduce the influx of inflammatory cells in the lower part of the spinal cord. GM-CSF antagonists are also able to reduce demyelination. For both parameters no histological scores of 2 or below could be observed in the control treatment, indicating the effectiveness of the treatment with MOR-GM.

In summary, this results demonstrates the effectiveness of GM-CSF antagonists in the treatment of multiple sclerosis.

### Example 3

### Therapeutic Effectiveness of a GM-CSF Specific Antibody Comprising SEQ ID NOs. 3 or 4

Example 2 is repeated. As GM-CSF antagonist, a GM-CSF specific antibody comprising an amino acid sequence of a heavy chain variable region as depicted in SEQ ID No.:1 or comprising an amino acid sequence of a light chain variable region as depicted in SEQ ID No.:2 is used. Another species than mouse may be used, in particular a species to which the antibody used in this experiment is cross reactive. Preferably the animal species used in this experiment is rat.

The animals, e.g. rat, treated with the isotype control antibody show significant increased signs of EAE as compared to the animals which received a GM-CSF specific antibody comprising an amino acid sequence of a heavy chain variable region as depicted in SEQ ID No.:1 or comprising an amino acid sequence of a light chain variable region as depicted in SEQ ID No.:2. This demonstrates the effectiveness of the antibodies in the treatment of EAE and MS.

### Example 4

### Therapeutic Effectiveness of a GM-CSF Specific Antibodies Comprising SEQ ID NOs. 5-20

Example 2 is repeated. As GM-CSF antagonist, a GM-CSF specific antibody comprising a H-CDR3 sequence selected from any one of SEQ ID NOs. 5-16 is used. Preferably, said antibodies additionally comprise the H-CDR1 sequence of SEQ ID NO. 16, and/or the H-CDR2 sequence of SEQ ID NO. 17, and/or the L-CDR1 sequence of SEQ ID NO. 18, and/or the L-CDR2 sequence of SEQ ID NO. 19), and/or the L-CDR3 sequence of SEQ ID NO. 20. Another species than mouse may be used, in particular a species to which the antibody used in this experiment is cross reactive. Preferably the animal species used in this experiment is rat.

The animals, e.g. rat, treated with the isotype control antibody show significant increased signs of EAE as compared to the animals which received a GM-CSF specific antibody according to the present example. This demonstrates the effectiveness of the antibodies in the treatment of EAE and MS.

### Example 5

### Therapeutic effectiveness of a GM-CSF specific antibodies comprising SEQ ID NOs. 21-26

Example 2 is repeated. As GM-CSF antagonist, a GM-CSF specific antibody comprising the L-CDR1 sequence of SEQ ID NO. 21, and/or the L-CDR2 sequence of SEQ ID NO. 22, and/or the L-CDR3 sequence of SEQ ID NO. 23, and/or the H-CDR1 sequence of SEQ ID NO. 24, and/or the H-CDR2 sequence of SEQ ID NO. 25, and/or the H-CDR3 sequence of SEQ ID NO. 26 is used. Preferably said antibody comprise all the CRDs of SEQ ID NOs. 21-26. Another species than mouse may be used, in particular a species to which the antibody used in this experiment is cross reactive. Preferably the animal species used in this experiment is rat.

The animals, e.g. rat, treated with the isotype control antibody show significant increased signs of EAE as compared to the animals which received a GM-CSF specific antibody according to the present example. This demonstrates the effectiveness of the antibodies in the treatment of EAE and MS.

### Example 6

### Therapeutic Effectiveness of a GM-CSF Specific Antibodies Comprising SEQ ID NOs. 46-56

Example 2 is repeated. As GM-CSF antagonist, a GM-CSF specific antibody comprising a H-CDR3 sequence selected from any one of SEQ ID NOs. 46-56 is used. Another species than mouse may be used, in particular a species to which the antibody used in this experiment is cross reactive.

The animals, e.g. a rhesus or cynomolgus monkey, treated with the isotype control antibody show significant increased signs of EAE as compared to the animals which received a GM-CSF specific antibody according to the present example. This demonstrates the effectiveness of the antibodies in the treatment of EAE and MS.

### Example 7

### Therapeutic Effectiveness of Antibodies Specific for the GM-CSF Receptor

Example 2 is repeated with the difference that a monoclonal antibody specific for the GM-CSF receptor is used instead of a monoclonal antibody specific for the GM-CSF.

As GM-CSF antagonist, a GM-CSF receptor specific antibody comprising an amino acid sequence of a H-CDR3 sequence depicted in any one of SEQ ID No's.:27-45 is used. Another species than mouse may be used, in particular a species to which the antibody used in this experiment is cross reactive. Preferably the animal species used in this experiment is rat.

The animals, e.g. rat, treated with the isotype control antibody show significant increased signs of EAE as compared to the animals which received a GM-CSF receptor specific antibody according to the present example. This demonstrates the effectiveness of the antibodies in the treatment of EAE and MS.

### Example 8

### Clinical Trial

Efficacy of the compounds of the present invention can be tested in a clinical trial for relapsing-remitting multiple sclerosis. The study population comprises patients (above 18 and below 55 years of age, both men and women) with a confirmed diagnosis of the relapsing and remitting form of multiple sclerosis (RRMS). Compounds are administered intravenously. Objective is to evaluate early efficacy of MOR-GM in patients with RRMS in a multicenter, double-blind, placebo-controlled, dose-ranging study.

Patients will be grouped into different treatment groups. The different treatment groups will receive either placebo, 0.75 mg, 1.5 mg or 3.0 mg MOR-GM every two weeks for the first two doses and thereafter once a month MOR-GM.

The clinical trial further confirms the efficacy of the GM-CSF antagonists of the present invention. Onset of multiple sclerosis after treatment with MOR-GM is clearly delayed as compared to treatment with placebo.

1. An antagonist of GM-CSF for use in the treatment or prophylaxis of multiple sclerosis.

2. The antagonist of claim 1, wherein said treatment or prophylaxis comprises the step of administering to a subject an effective amount of the antagonist of GM-CSF.

3. The antagonist of claim 2, wherein said subject is a human.

4. The antagonist of claim 2, wherein said subject is a rodent, such as a rat or a mouse.

5. The antagonist of claim 1, wherein said antagonist is an antibody specific for GM-CSF.

6. The antagonist of claim 5, wherein the variable heavy chain of said antibody specific for GM-CSF comprises the amino acid sequence of SEQ ID No.:3.

7. The antagonist of claim 5, wherein the variable light chain of said antibody specific for GM-CSF comprises the amino acid sequence of SEQ ID No.:4.

8. The antagonist of claim 1, wherein said antagonist is an antibody specific for the GM-CSF receptor.

9. The antagonist of claim 1, wherein said treatment or prophylaxis reduces the demyelination of the myelin sheet.

10. The antagonist of claim 1, wherein said treatment or prophylaxis reduces the influx of inflammatory cells into the spinal cord.

11. The antagonist of claim 1, wherein said treatment or prophylaxis reduces the proliferation of T cells.

12. The antagonist of claim 1, wherein said treatment or prophylaxis reduces the release of IL17 by T cells.

13. The antagonist of claim 1, wherein said treatment or prophylaxis delays the onset of multiple sclerosis.

14. The antagonist of claim 2, wherein said antagonist of GM-CSF is administered subcutaneously or intraspinally.


SEQUENCE LISTING (57 sequences)

SEQ ID NO: 1 - protein, 140 aa, Artificial Sequence
  Description of Artificial Sequence Synthetic polypeptide
MELIMLFLLSGTAGVHSEVQLQQSGPELVKPGASVKISCKASGYTFTDYNIHWVKQSHGKSLDWIGYIAPYSGGTGYNQEFKNRATLTVDKSSSTAYMELRSLTSDDSAVYYCARRDRFPYYFDYWGQGTTLRVSSVSGS

SEQ ID NO: 2 - protein, 150 aa, Artificial Sequence
  Description of Artificial Sequence Synthetic polypeptide
MGFKMESQIQVFVYMLLWLSGVDGDIVMIQSQKFVSTSVGDRVNITCKASQNVGSNVAWLQQKPGQSPKTLIYSASYRSGRVPDRFTGSGSGTDFILTITTVQSEDLAEYFCQQFNRSPLTFGSGTKLELKRADAAPTVSIFPPSSKGEF

SEQ ID NO: 3 - protein, 115 aa, Artificial Sequence
  Description of Artificial Sequence Synthetic polypeptide
QVQLVESGGGLVQPGGSLRLSCAASGFTFSSYWMNWVRQAPGKGLEWVSGIENKYAGGATYYAASVKGRFTISRDNSKNTLYLQMNSLRAEDTAVYYCRFGTDFWGQGTLVTVSS

SEQ ID NO: 4 - protein, 106 aa, Artificial Sequence
  Description of Artificial Sequence Synthetic polypeptide
DIELTQPPSVSVAPGQTARISCSGDSIGKKYAYWYQQKPGQAPVLVIYKKRPSGIPERFSGSNSGNTATLTISGTQAEDEADYYCSAWGDKGMVFGGGTKLTVLGQ

SEQ ID NO: 5 - protein, 10 aa, Artificial Sequence
  Description of Artificial Sequence Synthetic peptide
SGLIFDYWLD

SEQ ID NO: 6 - protein, 10 aa, Artificial Sequence
  Description of Artificial Sequence Synthetic peptide
SGLIIDALSP

SEQ ID NO: 7 - protein, 10 aa, Artificial Sequence
  Description of Artificial Sequence Synthetic peptide
TSLMSIYFDY

SEQ ID NO: 8 - protein, 10 aa, Artificial Sequence
  Description of Artificial Sequence Synthetic peptide
SGLLFLYFDY

SEQ ID NO: 9 - protein, 10 aa, Artificial Sequence
  Description of Artificial Sequence Synthetic peptide
SGLINLGMHP

SEQ ID NO: 10 - protein, 10 aa, Artificial Sequence
  Description of Artificial Sequence Synthetic peptide
SGLIFDALRD

SEQ ID NO: 11 - protein, 10 aa, Artificial Sequence
  Description of Artificial Sequence Synthetic peptide
SGLIFDKLTS

SEQ ID NO: 12 - protein, 10 aa, Artificial Sequence
  Description of Artificial Sequence Synthetic peptide
SGLINLHFDT

SEQ ID NO: 13 - protein, 10 aa, Artificial Sequence
  Description of Artificial Sequence Synthetic peptide
STHFSAYFDY

SEQ ID NO: 14 - protein, 10 aa, Artificial Sequence
  Description of Artificial Sequence Synthetic peptide
SGLIMDKLDN

SEQ ID NO: 15 - protein, 10 aa, Artificial Sequence
  Description of Artificial Sequence Synthetic peptide
SGLIIDNLNP

SEQ ID NO: 16 - protein, 10 aa, Artificial Sequence
  Description of Artificial Sequence Synthetic peptide
SGLIAVYFDY

SEQ ID NO: 17 - protein, 17 aa, Artificial Sequence
  Description of Artificial Sequence Synthetic peptide
WLNPYSGDTNYAQKFQG

SEQ ID NO: 18 - protein, 11 aa, Artificial Sequence
  Description of Artificial Sequence Synthetic peptide
RASQNIRNILN

SEQ ID NO: 19 - protein, 7 aa, Artificial Sequence
  Description of Artificial Sequence Synthetic peptide
AASNLQS

SEQ ID NO: 20 - protein, 9 aa, Artificial Sequence
  Description of Artificial Sequence Synthetic peptide
QQSYSMPRT

SEQ ID NO: 21 - protein, 12 aa, Artificial Sequence
  Description of Artificial Sequence Synthetic peptide
RASHRVSSNYLA

SEQ ID NO: 22 - protein, 7 aa, Artificial Sequence
  Description of Artificial Sequence Synthetic peptide
GASNRAT

SEQ ID NO: 23 - protein, 9 aa, Artificial Sequence
  Description of Artificial Sequence Synthetic peptide
QQYASSPVT

SEQ ID NO: 24 - protein, 10 aa, Artificial Sequence
  Description of Artificial Sequence Synthetic peptide
GYIFPTFALH

SEQ ID NO: 25 - protein, 16 aa, Artificial Sequence
  Description of Artificial Sequence Synthetic peptide
SINTASGKTKFSTKFQ

SEQ ID NO: 26 - protein, 13 aa, Artificial Sequence
  Description of Artificial Sequence Synthetic peptide
DRFQNIMATILDV

SEQ ID NO: 27 - protein, 11 aa, Artificial Sequence
  Description of Artificial Sequence Synthetic peptide
VGSFSGIAYRP

SEQ ID NO: 28 - protein, 11 aa, Artificial Sequence
  Description of Artificial Sequence Synthetic peptide
VGSFSGPALRP

SEQ ID NO: 29 - protein, 11 aa, Artificial Sequence
  Description of Artificial Sequence Synthetic peptide
VGSFSPPTYGY

SEQ ID NO: 30 - protein, 11 aa, Artificial Sequence
  Description of Artificial Sequence Synthetic peptide
VGSFSGYPYRP

SEQ ID NO: 31 - protein, 11 aa, Artificial Sequence
  Description of Artificial Sequence Synthetic peptide
VGSFSPLTLGL

SEQ ID NO: 32 - protein, 11 aa, Artificial Sequence
  Description of Artificial Sequence Synthetic peptide
VGSFSGPVYGL

SEQ ID NO: 33 - protein, 11 aa, Artificial Sequence
  Description of Artificial Sequence Synthetic peptide
VGSFSPPAYRP

SEQ ID NO: 34 - protein, 11 aa, Artificial Sequence
  Description of Artificial Sequence Synthetic peptide
VGSFSPVTYGL

SEQ ID NO: 35 - protein, 11 aa, Artificial Sequence
  Description of Artificial Sequence Synthetic peptide
VGSFSGLAYRP

SEQ ID NO: 36 - protein, 11 aa, Artificial Sequence
  Description of Artificial Sequence Synthetic peptide
VGSFSPITYGL

SEQ ID NO: 37 - protein, 11 aa, Artificial Sequence
  Description of Artificial Sequence Synthetic peptide
VGSFSGWAFDY

SEQ ID NO: 38 - protein, 11 aa, Artificial Sequence
  Description of Artificial Sequence Synthetic peptide
VGSFSGWAFDY

SEQ ID NO: 39 - protein, 11 aa, Artificial Sequence
  Description of Artificial Sequence Synthetic peptide
LGSVTAWAFDY

SEQ ID NO: 40 - protein, 11 aa, Artificial Sequence
  Description of Artificial Sequence Synthetic peptide
AGSIPGWAFDY

SEQ ID NO: 41 - protein, 11 aa, Artificial Sequence
  Description of Artificial Sequence Synthetic peptide
VGSFSPLTMGL

SEQ ID NO: 42 - protein, 11 aa, Artificial Sequence
  Description of Artificial Sequence Synthetic peptide
VGSFSPLTMGL

SEQ ID NO: 43 - protein, 11 aa, Artificial Sequence
  Description of Artificial Sequence Synthetic peptide
VGSFSGPALHL

SEQ ID NO: 44 - protein, 11 aa, Artificial Sequence
  Description of Artificial Sequence Synthetic peptide
VGSVSRITYGF

SEQ ID NO: 45 - protein, 11 aa, Artificial Sequence
  Description of Artificial Sequence Synthetic peptide
VGSFSPLTLGL

SEQ ID NO: 46 - protein, 11 aa, Artificial Sequence
  Description of Artificial Sequence Synthetic peptide
EGGYSYGYFDY

SEQ ID NO: 47 - protein, 9 aa, Artificial Sequence
  Description of Artificial Sequence Synthetic peptide
DKWLDGFDY

SEQ ID NO: 48 - protein, 9 aa, Artificial Sequence
  Description of Artificial Sequence Synthetic peptide
DRWLDAFDI

SEQ ID NO: 49 - protein, 9 aa, Artificial Sequence
  Description of Artificial Sequence Synthetic peptide
APYDWTFDY

SEQ ID NO: 50 - protein, 9 aa, Artificial Sequence
  Description of Artificial Sequence Synthetic peptide
DRWLDAFEI

SEQ ID NO: 51 - protein, 9 aa, Artificial Sequence
  Description of Artificial Sequence Synthetic peptide
QRYYYSMDV

SEQ ID NO: 52 - protein, 9 aa, Artificial Sequence
  Description of Artificial Sequence Synthetic peptide
RPWELPFDY

SEQ ID NO: 53 - protein, 11 aa, Artificial Sequence
  Description of Artificial Sequence Synthetic peptide
NGDYVFTYFDY

SEQ ID NO: 54 - protein, 10 aa, Artificial Sequence
  Description of Artificial Sequence Synthetic peptide
FGYFGYYFDY

SEQ ID NO: 55 - protein, 9 aa, Artificial Sequence
  Description of Artificial Sequence Synthetic peptide
DPYTSGFDY

SEQ ID NO: 56 - protein, 10 aa, Artificial Sequence
  Description of Artificial Sequence Synthetic peptide
EDTAMDYFDY

SEQ ID NO: 57 - protein, 5 aa, Artificial Sequence
  Description of Artificial Sequence Synthetic peptide
DYLLH